(19) United States
(12) Patent Application Publication
(10) Pub. No.: US 2023/0225915 A1
(43) Pub. Date: Jul. 20, 2023

(54) PATIENT SUPPORT APPARATUS COVER

(71) Applicant: Stryker Corporation, Kalamazoo, MI (US)

(72) Inventors: Justin Raymond, Jackson, MI (US); Patrick Lafleche, Kalamazoo, MI (US); James K. Galer, Byron Center, MI (US); Maria del Mar Davila, Fishers, IN (US)

(21) Appl. No.: 18/011,638

(22) PCT Filed: Sep. 28, 2021
(86) PCT No.: PCT/US2021/052313

§ 371 Date: Dec. 20, 2022

Related U.S. Application Data
(60) Provisional application No. 63/093,833, filed on Oct. 20, 2020.

(51) Int. Cl. A61F 13/511; A61G 7/057; A61F 13/513

(52) U.S. Cl. CPC A61F 13/5116; A61G 7/05769; A61F 13/513; A61F 2013/15056

(57) ABSTRACT

A cover for covering a patient support apparatus cushion includes a top cover layer, a lower cover layer configured to join with the top cover layer for enclosing a cushion there between, and an ePTFE membrane between the top cover layer and the lower cover layer to form a liquid barrier between the top cover layer and the cushion. In one form, the ePTFE membrane is attached to the top cover layer. In another form, the ePTFE membrane is unattached to the top cover layer.

### TECHNICAL FIELD AND BACKGROUND

The present disclosure relates covers for patient support apparatuses. When supporting patients on patient support apparatuses there is potential for liquid spills, such as urine or blood spills. If the liquid penetrates the cover, the liquid can enter the underlying cushion, eventually resulting in the need for the cushion to be replaced.

To avoid these potential problems, absorbent pads are often placed under the patient. However, the pads have to monitored and replaced regularly, especially in the case of an incontinent patient. If the pads are not changed frequently enough, it may lead to the patient being exposed to high levels of moisture, which can exacerbate or cause tissue injury—potentially leading to the formation of pressure ulcers or sores. Additionally, these pads may interfere with a patient's immersion into the support surface, especially into mattresses that have been more recently designed to better distribute pressure on a patient's skin by allowing the patient to immerse or sink into the mattress. Again, when the pressure distribution is impaired, this also can lead to the formation of pressure ulcers or sores for patients with compromised skin.

More recently, covers have been formed from impermeable materials to protect the cushion from coming into contact with a liquid. These more recent covers have been made from a flexible fabric with a flexible liquid proof coating, such as polyvinyl chlorides (“PVCs”) or polyurethanes. Over time, however, these covers wear and the coatings can lose their ability to prevent entry of a liquid into the cushion. While some failure modes are obvious, such as large cracks or tears in the cover, or a visible delamination of the coating, other failure modes are less observable.

### SUMMARY

Accordingly, the present disclosure provides several covers that are configured to reduce the chances of liquid intrusion into the underlying cushion and/or provide a visual indication when the cover has been breached so that the cover can be replaced.

In one embodiment, a cover for covering a patient support apparatus cushion includes a top cover layer, a lower cover layer configured to join with the top cover layer for enclosing a cushion there between, and an ePTFE membrane between the top cover layer and the lower cover layer to form a liquid barrier between the top cover layer and the cushion.

In one embodiment, the ePTFE membrane is attached to the top cover layer.

For example, when the top cover layer comprises first and second layers, with the first layer forming an exterior surface of the cover, the ePTFE membrane is then secured to the second layer. In a further aspect, the ePTFE membrane may be laminated to the second layer.

In another embodiment, the ePTFE membrane is unattached to the top cover layer.

In yet another embodiment, the cover further includes an inner layer, with the ePTFE membrane secure to the top cover layer between the inner layer and the second layer.

In any of the above, the ePTFE membrane may be configured as a sleeve or a sock with at least two open sides for receiving the cushion therein.

According to another embodiment, a mattress for a patient support apparatus includes a cushion, a top cover layer, a lower cover layer configured to join with the top cover layer for enclosing the cushion there between, and an ePTFE membrane between the top cover layer and the lower cover layer to form a liquid barrier between the top cover layer and the cushion.

In one embodiment, the ePTFE membrane is attached to the top cover layer or may be unattached to the top cover layer.

In yet other aspects, the top cover layer comprises first and second layers, with the first layer forming an exterior surface of the cover, and with the ePTFE membrane secure to the second layer. For example, the ePTFE membrane may be laminated or spot-welded to the second layer.

In yet another embodiment, the ePTFE membrane is configured as a sock enclosing the cushion therein.

In any of the above covers or in a conventional cover, the cover may include an intrusion indicator configured to indicate moisture or liquid intrusion through the cover, where the instruction detection is configured to indicate a change in moisture, wetness, or pH.

In one embodiment, the intrusion indicator is secured the top cover layer.

In any of the above, the intrusion indicator may comprise an ink printed on or in the top cover layer.

Alternately, in any of the above, the intrusion indicator may comprise a tape secured to the top cover layer between the top layer and the cushion.

For example, when formed from a tape, the tape may extend exteriorly of the cover.

In yet another embodiment, the top cover layer may include a transparent window, with the intrusion indicator located behind and viewable through the window.

In any of the above, the intrusion indicator may comprise a hydrochromic ink or hydrochromic tape. Thus, when the ink or tape has been exposed to liquid, the color of ink or tape will provide an indication that liquid has passed through the cover into the space inside the cover.

Before the embodiments of the invention are explained in more detail below, it is to be understood that the invention is not limited to the details of operation or to the details of construction and the arrangement of the components set forth in the following description or illustrated in the drawings. The invention may be implemented in various other embodiments and is capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a perspective view of one embodiment of a patient support apparatus including a cover for covering a cushion in the form of a mattress;

FIG. 1A is a perspective view of one embodiment of the cover and cushion;

FIG. 1B is a perspective view of one embodiment of the cushion;

FIG. 2 is an enlarged cross-section of the top portion of the cover;

FIG. 2A is an enlarged cross-section another embodiment of the top portion of the cover;

FIG. 2B is an enlarged cross-section of yet another embodiment of the top portion of the cover;

FIG. 3 is an exploded perspective view of one embodiment of the cover;

FIG. 4 is an exploded perspective view of another embodiment of the cover;

FIG. 5 is an exploded perspective view of another embodiment of the cover;

FIG. 6 is an enlarged schematic diagram of another embodiment of the cover with an intrusion indicator;

FIG. 7 is an enlarged schematic diagram of another embodiment of the cover with an intrusion indicator;

FIG. 8 is an enlarged schematic diagram of another embodiment of the cover with an intrusion indicator;

FIG. 9 is an enlarged schematic diagram of another embodiment of the cover with an intrusion indicator; and

FIG. 10 is an enlarged schematic diagram of another embodiment of the cover with an intrusion indicator.

### DESCRIPTION OF THE EMBODIMENTS

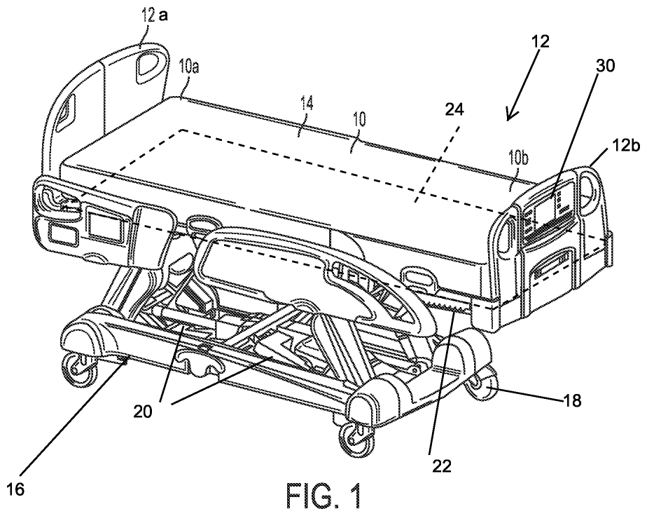

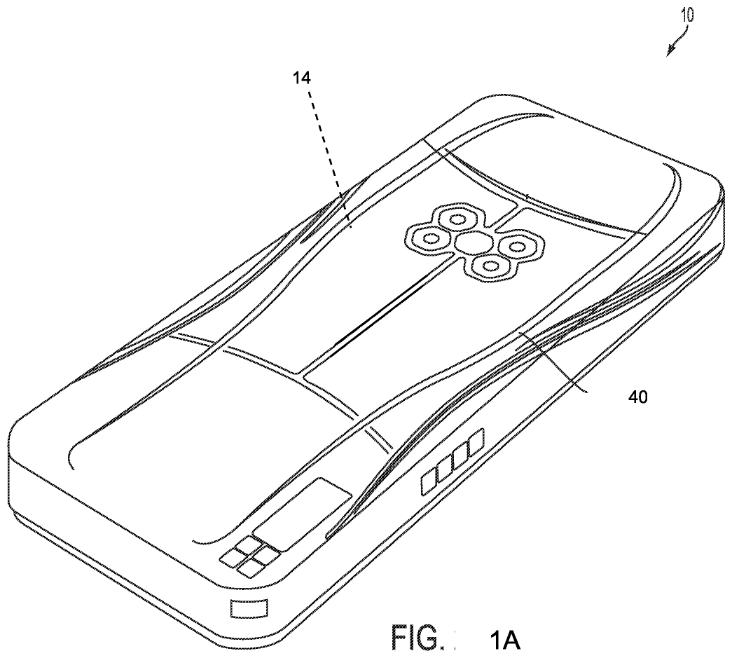

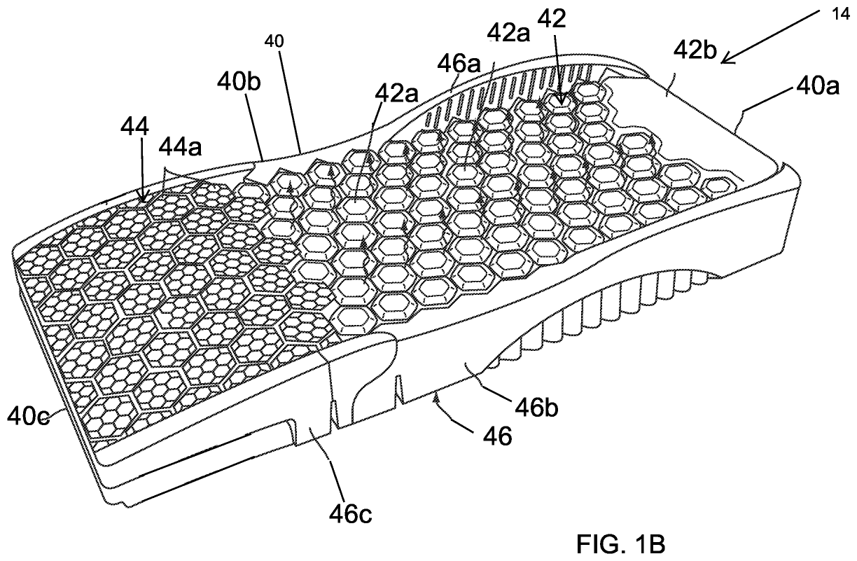

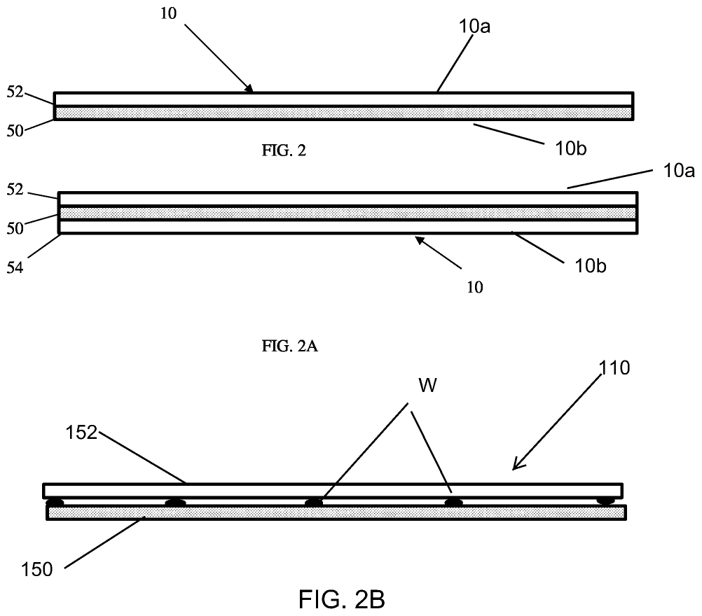

Referring to FIGS. 1 and 2, the numeral 10 generally designates a cover. As will be more fully described below, cover 10 is configured to cover a cushion 14 for a patient support apparatus 12. While described as a “patient” support apparatus, it should be understood that “patient” is to be construed broadly to include not only people undergoing medical treatment but also invalids and other persons, such as long-term care persons, who may need assistance or care but who may or may not be undergoing medical treatment. Further, although the particular form of the patient support apparatus illustrated in FIG. 1 is a bed adapted for use in a hospital or other medical setting, it will be understood that the patient support apparatus could, in different embodiments, be an EMS cot, a stretcher, a recliner, a wheelchair, operating room (OR) table or any other mobile structure capable of supporting a patient in a healthcare environment, as well as in a residential setting, such as a residential bed.

Referring to FIG. 1A, cover 10 is configured to cover cushion 14, which is supported on patient support apparatus 12. For further details of an exemplary patient support apparatus reference is made to the below description and the referenced applications and patents. For example, cushion 14 may be formed from one or more gel cushioning layers, pneumatic bladders, and/or foam or a combination of two or more. In the case of pneumatic bladders, the bladders may be powered or non-powered depending on the application and features incorporated therein.

In the illustrated embodiment, with reference to FIG. 1B, cushion 14 is the form of a mattress 40 that includes a bladder layer 42 and a gel layer 44, which are both supported on a foam crib 46. Further, in the illustrated embodiment bladder layer 42 is formed from a plurality of pods 42a, which are arranged laterally and longitudinally along the mattress from or near the head end 40a of mattress 40 to a medial portion 40b of the mattress 40 past the sacrum area of a patient supported thereon. Optionally, the bladder layer also includes a larger bladder 42b at the head end for support the head of a patient. The gel layer is similarly formed from gel pods in the form of footings 44a, which extend from the bladder layer to the foot end 40c of the mattress.

The foam crib 46 includes a base wall (not shown) and a pair of opposed side walls 46a and 46b that extend upwardly from the base wall and between which the bladder layer and the gel layer are supported. To facilitate access to beneath the foot end of the mattress, the foot end of the crib may include a hinged section 46c. For examples of a suitable cushion or mattress, reference is made to U.S. Pat. No. 9,420,895, filed on Sep. 5, 2013 entitled PATIENT SUPPORT (P405E) and U.S. Pat. No. 9,468,307, U.S. Ser. No. 13/836,813, filed Mar. 15, 2013, entitled INFLATABLE MATTRESS AND CONTROL METHODS (143667.150316(P400A)). However, it should be understood, as noted, that the cushion or mattress may take on a variety of different forms.

As noted above, cover 10 is configured to reduce the chances of liquid intrusion into the underlying cushion 14, such as mattress 40, and/or provide an indication when the cover has been breached. Further, as will be more fully described below, cover 10 may be configured to replace a conventional fire sock, which is typically provided between mattress covers and their underlying mattresses, and consequently provide a single cover to cover the mattress, which can reduce if not eliminated interference with a myriad of functions, such as moisture vapor transmission MVTR, envelopment, and/or immersion.

As will be more fully described below, expanded Polytetrafluoroethylene ePTFE is incorporated into cover 10 to improve its breathability but limit, if not prevent, liquid intrusion through the cover 10. ePTFE is often formed as a membrane and are formed through the expansion of the linear polymer made of carbon and fluorine molecules, which have their nodes connected by fibrils and are non-permeable to liquid. This allows for a high moisture vapor transmission rate (MVTR), which is a measure of the material's breathability in terms of the grams of moisture vapor being transmitted through a membrane over time and within an area. In essence, ePTFE has a selective membrane that allows gas molecules to pass but repels liquid and particles. Therefore, moisture vapor can be transmitted through the membrane to control the patient's microclimate but not allow sweat to stay on the skin and degrade it. On the other hand, liquid molecules including blood and urine would not be able to pass through the membrane and, therefore, would not reach the mattress.

In one embodiment, referring to FIG. 2, the top portion of cover 10 is formed from an ePTFE membrane 50 and a reinforcing layer 52, such as a layer of polyester, nylon, or urethane, including an engineered polyurethane (PU), such as Dartex. Due to ePTFE's porous matrix, the reinforcing layer forms the top surface of the cover 10, for example, the patient facing side 10a of the cover, with the ePTFE membrane forming the inner surface, such as the cushion facing side 10b, which faces the core cushion 14. Optionally, ePTFE membrane 50 may be laminated with the reinforcing layer 52 or cast coated with a coating, such as polyurethane, which forms the reinforcing layer 52.

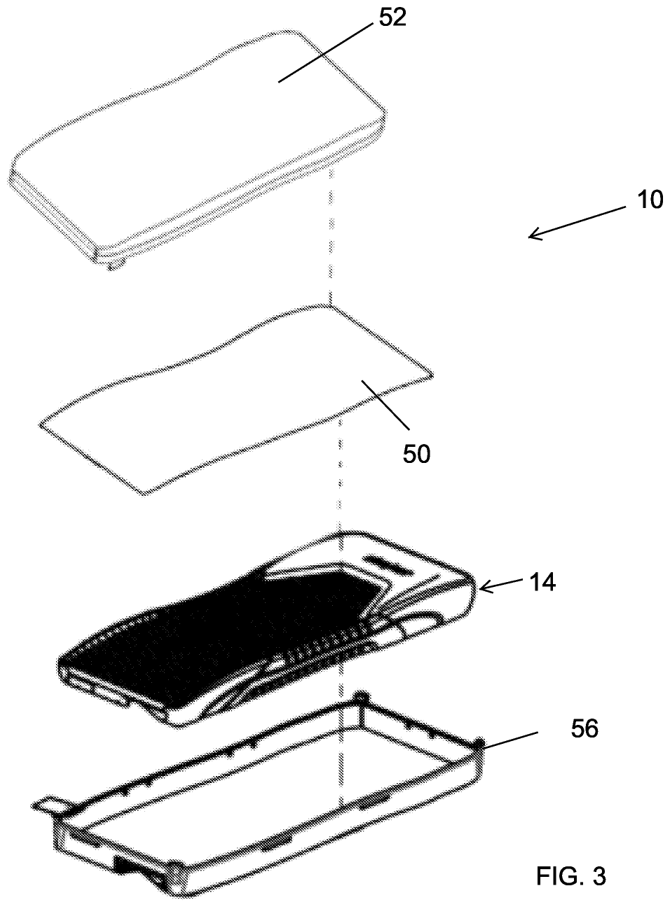

In one embodiment, as noted, reinforcing layer 52 may form the top cover layer, such as shown in FIG. 3. Further, ePTFE membrane 50 may laminated directly to the underside of layer 52, which may comprise an already fabricated polyurethane coated fabric top cover, like Dartex, which is polyurethane cast-coated onto a nylon and/or polyester layer, which forms reinforcing layer 52. In this manner, cover 10 then includes a nylon or polyester substrate with a polyurethane coating on its top side and ePTFE membrane 50 on its lower, or middle, side as noted below. Top cover layer 52 is then joined to a lower or bottom layer 56 to thereby enclose the cushion 14 there between. For example, top cover layer 52 may be joined to lower layer 56 by a zipper, including a full perimeter zipper, to provide a fluid tight seal.

Referring to FIG. 2A, an additional layer 54 may be laminated or coated onto the ePTFE membrane 50, for example another polyurethane coating or another layer or substrate, such as polyester, nylon, or urethane, which forms the cushion facing side 10b of cover 10. Thus, the ePTFE membrane 50 may be laminated between two layers, such as layers 52 and 54.

In one embodiment, layer 54 forms a carrier for the ePTFE to compensate for some of the mechanical limitations of ePTFE. For example, layer 54 may be formed from a knit or non-woven material, which is then laminates with the ePTFE membrane, and which reinforces the ePTFE membrane.

In another embodiment, referring to FIG. 2B, cover 110 may be formed from an ePTFE membrane 150, similar to membrane 50, and a top layer 152, such as a polyester, nylon, or urethane layer, including an engineered polyurethane (PU), such as Dartex. The two layers are then secured together, for example, by spot welds or glue or stitches W or the like, and then joined with lower or bottom layer by, for example, a zipper, such as a full perimeter zipper, to provide a fluid tight seal.

For example, ePTFE membrane 150 may be joined to the top cover layer 152 around and inside the outer perimeter of perimeter top cover layer 152 or may be joined at intermediate points across the width and length of the top cover layer 152. In this manner, similar to cover 10, cover 110 may be formed using a conventional cover, such as Dartex, as the top side of cover 110. Further, only the top side of the cushion is protected from liquid intrusion.

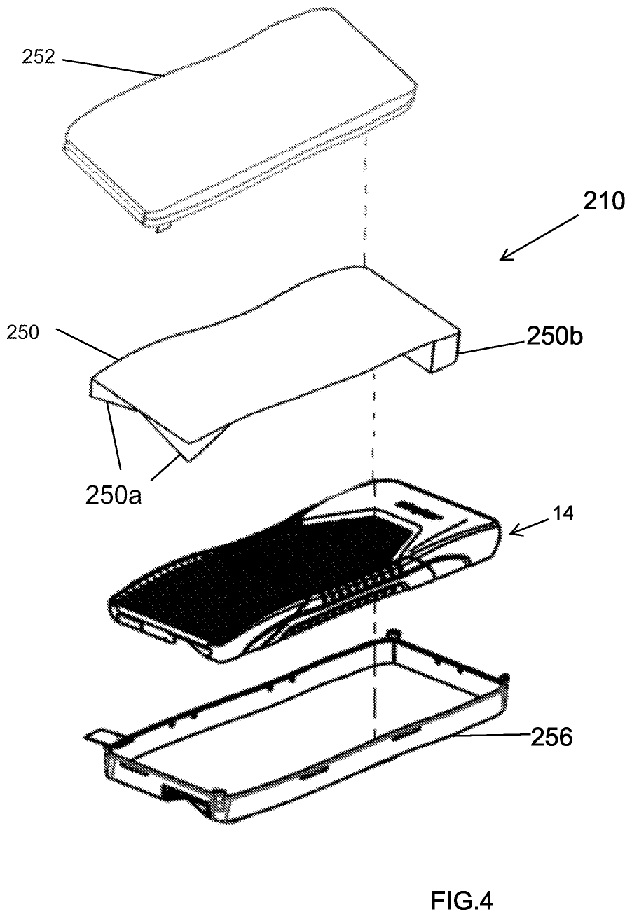

Referring to FIG. 4, the numeral 210 designates another embodiment of the cover. Cover 210 is formed from an ePTFE member 250, similar to above embodiments, and a top cover layer 252, which again may be formed from a conventional cover, such as a Dartex cover, which is then joined with a lower or bottom layer 256 by, for example, a full perimeter zipper, to provide a fluid tight seal.

In the illustrated embodiment, membrane 250 is detached, i.e., not attached, to top cover layer 252 and instead is held in place about cushion 14 by straps 250a, such as elastic/spandex straps, attached to membrane 250, such as at or near its opposed corners, which stretch over and secure the membrane to the four corners of the underlying cushion.

Optionally, straps 250a may be attached at or near the corners on one end, such as the foot end, and membrane 250 may be formed with an extension or attached to a panel of material that forms a pocket 250b on its opposed end, such as its head end, that envelops the end of the cushion (both corners) to secure member 250 about cushion 14. Pocket 250b may be provided with elastic secured around its free edges, like a fitted sheet, to better secure the membrane to the cushion 14. Alternately, or in addition, the pocket may be formed from an elastic material. Optionally, pockets 250b may be provided at both ends in lieu of straps 250a. Further, pockets 250b may be formed from triangular shaped extensions of the membrane or triangular shaped panels that engage just the corners of the underlying cushion

Additionally, as noted, pocket or pockets 250b may be formed from extensions of the membrane or formed from panels that are secured to the membrane by stitching or welding or by removable fasteners, such as zippers, snaps, buttons, or VELCRO strips.

In this manner, membrane 250 is a separate component from the mattress top cover layer. Thus potentially, only the top side of the cushion is protected from liquid intrusion.

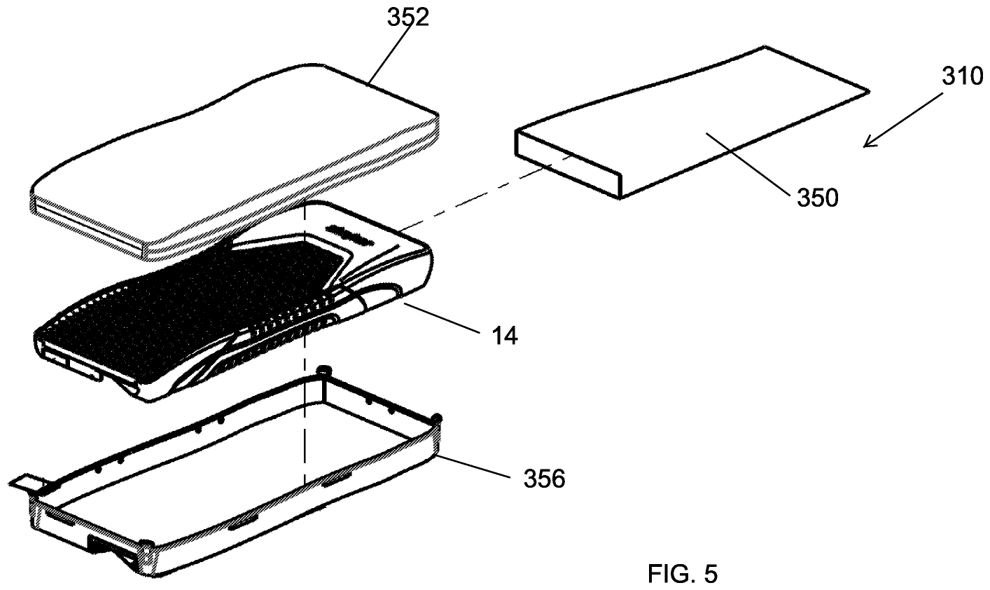

Referring to FIG. 5, in yet another embodiment, cover 310 is formed from a membrane 350, which is configured as a sleeve or sock, a top cover layer 352, and a lower or bottom cover layer 356. Again, top cover layer 352 is joined with lower or bottom cover layer 356, for example, via a zipper similar to the previous embodiments, to provide a fluid tight seal.

Membrane 350 may completely encase cushion 14 and, further, be a separate component from the top cover layer 352 similar to top cover layer 252. For example, membrane 350 may be folded over and joined, such as by stitching or welding or adhesive bonding, along one or two sides leaving two or one side open for receiving cushion 14 therein. Alternately, the sleeve or sock may be formed from two ePTFE membranes joined, such as by stitching, welding, or adhesive bonding, on 2 or 3 sides with the 3rd and/or 4th side open to receive cushion 14 therein. In another embodiment, the sock or sleeve may be formed with its upper layer formed from the membrane, and its lower layer under the cushion, which is joined with the membrane formed from another material, such as nylon, PU or other suitable flexible fabrics.

Optionally, after the cushion is inserted, the open side or sides (open when being assembled) of the sleeve or sock may be closed in a fluid-tight method. For example, the membrane 350 may be sized so its open side(s) extends beyond the cushion so that it can be folded and tucked under the cushion 14. Alternately, membrane 350 may have an extended flap on one side (or both sides) that can be folded and tucked under the cushion 14.

In another embodiment, the open side may be closed by zipper or snaps or the like, optionally with an extended lip that extends over the seam (formed by the zipper or snaps or the like), to form a watershed cover.

Consequently, based on its properties, when closed on its open side(s) membrane 350 may form a fire sock and, hence, eliminate the need for a separate fire sock.

As will be more fully described below, any of the above covers 10, 110, 210, and 310, as well as a conventional cover, may comprise a cover 410 with an intrusion indicator in the space between the cushion and the top cover layer that provides a visual indication when a liquid breach through the cover has occurred.

The intrusion indicator can be located anywhere under the cover. For example, intrusion indicator 416 may be located in a location on the cover where it is easily visible to a caregiver. For example, locating the intrusion indicator (where it is visible though the cover or exteriorly of the cover, as more fully described below), such as at the foot end or at the sacrum region of the cover, for example including at the upper edge of the cover, would allow a care giver to quickly check the cover for liquid intrusion without having to move the patient.

In the illustrated embodiment, cover 410 includes a top cover layer 412, a lower or bottom cover layer 414, which are joined together about a cushion 14, such as described above, and an intrusion indicator 416. As more fully described below, intrusion indicator 416 is configured to provide an indication of when a fluid comes into contact with intrusion indicator 416 through cover 410 so that a caregiver can be notified when fluid has breached the cover 410, and optionally before it enters into cushion.

The intrusion indicator 416 can be incorporated in or on the cover 410 in a variety of ways. For example, the intrusion indicator 416 may be printed on or adhered to one of the layers as noted below. For example, the intrusion indicator 416 can be sprayed or directly or indirectly printed onto the respective layer using, for example, dye sublimation printing. The intrusion indicator 416 may not be a discrete or separate layer, and further may be formed within or on the inner layer when printed. Similarly, the intrusion indicator 416 may be formed from a contrasting color before it changes color so that a user can see where the indictor is even before changing color and then is selected so that it changes to yet another contrasting color from both the cover and its original color.

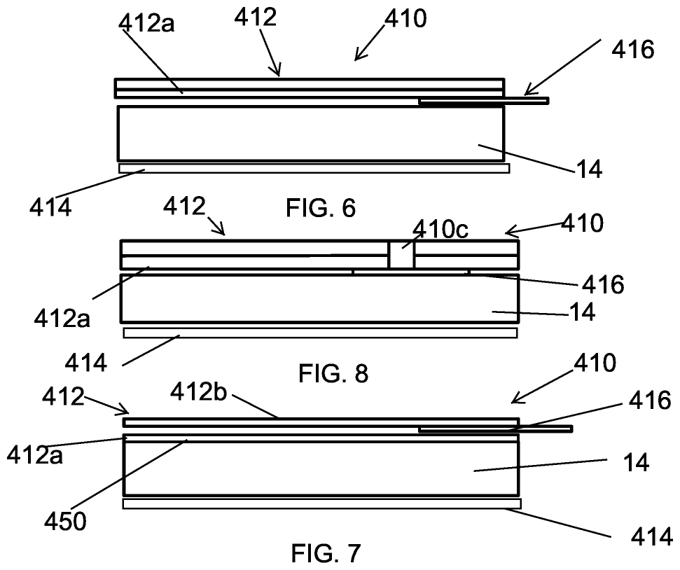

Referring to FIG. 6, in one embodiment, intrusion indicator 416 may be located on the inner side of cover 410, for example, between top cover layer 412 and cushion 14 so that it is located in the space between the top cover layer and the cushion. Optionally, when cover 410 is formed from more than two layers, for example a coated nylon or coated polyurethane layer, intrusion indicator 416 may be located between the two layers or on the inside surface of the inner layer 412a, such as the nylon or polyurethane layer.

Alternately, as shown in FIG. 7, when cover 410 incorporates an ePTFE membrane 450, as described above, the inner layer may be formed by the member 450, and the intrusion indicator 416 may be located between inner layer 412a, such as membrane 450, and top layer 412b of cushion 14.

For example, intrusion indicator 416 may comprise a tape or ink which is applied to the cover, for example, to the inside surface or intermediate surface, which contains a chemical that changes color when exposed to moisture, wetness and/or pH and/or specific fluid products, such as blood or urine. Some suitable indicators may include inks, such as hydrochromic ink available from Colourchange or Thermometer Site (both divisions of LCR Hallcrest) or from TMC Hallcrest or from Matsui Ink or inks available from CTI; a wetness indicator adhesive, available from H.B. Fuller; or hydrochromic tape from 3M. Being able to see the intrusion indicator 416 allows a caregiver to be notified when cover 410 is ready for replacement.

When in the form of a tape, the tape may be applied to (for example by welding or gluing) or integrally formed with the cover or releasable attached to the cover, for example, by a releasable fastener, such as snaps, hook and loop fasteners, or by a tether, such as a lanyard.

In one embodiment, as shown in FIGS. 6 and 7, when intrusion indicator 416 is formed from a tape, the tape may extended from the cover, through for example, the side of the cover, for example through a seam in the cover, so that a portion of the tape is external and visible from outside the cover. In this manner, when the portion of the tape inside the cover is exposed to a fluid, the whole tape will change color including that portion that extends form the cover. When extended from the inside of the cover to the outside of the cover, the external portion of the tape may be enclosed and protected with a transparent or translucent coating or covering (e.g. a sheath or a transparent or translucent panel that secures it to the outside surface of the cover) to avoid a false reading due to external moisture, wetness etc, which it may encounter due to normal use of the mattress.

Alternately, as best seen in FIG. 8, in one embodiment, cover 410 may include a window 410c formed from a transparent or translucent material, with the intrusion indicator located beneath the window so that its change can be visible through the cover. For example, the window may be formed from a clear polyurethane or a clear vinyl.

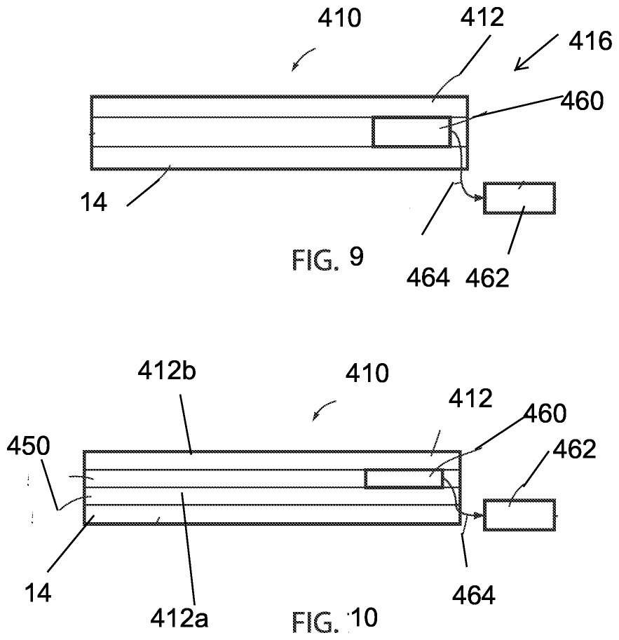

Referring to FIGS. 9 and 10, in another embodiment of the cover, intrusion indicator comprises an electrical sensor 460, such as a circuit that generates a signal when moisture, a liquid or a pH change is detected. For example, the signal can be transmitted to a device 462 external to cover 410, such as a user interface, including a user interface on the patient support apparatus or a hand held user interface, such as tablet, phone, or other communication device carried or worn by a caregiver, which generates a message, either text or an audible or visual message, such as a sound or light, to indicate to a caregiver that the cover has been breached.

Sensor 460 maybe located between top cover layer 412 and cushion 14 (FIG. 9) or when cover has multiple layers, such as any of the above embodiments with an inner layer, such as an inner layer formed by an ePTFE membrane 450, may be located between inner layer 412a and top cover layer 412b.

For example, sensor 460 may be formed by a pair of capacitor plates, which are then coupled to a processor that detects when a current passes between the capacitor plates, which indicates when moisture or a liquid is present, which then generates the signal to indicate when moisture, liquid or a change on pH is detected. The signal from the processor may be transmitted via a link 464, such as wireless or hardwired link, to device 462. Other suitable sensors may include battery free moisture sensors, such as a battery free RFID chip. Further, as noted, the sensors may measure pH, and based on the pH or change in pH, the processor can detect when a liquid is present.

As noted above, the above covers are suitable for covering a wide variety of cushions, including a mattress, and are suitable for use on a variety of patient support apparatus. In the illustrated embodiment, and as shown in FIG. 1, patient support apparatus 12 may be configured as a hospital bed and includes a base 16 with a plurality of wheels 18, one or more elevation adjustment mechanisms 20 supported on base 16, a frame or litter 22 supported on elevation adjustment mechanisms 20, and a patient support deck 24 (shown in phantom) supported on frame 22. Patient support apparatus 12 also includes a headboard 12a and a footboard 12b. Either or both of headboard 12a and footboard 12b may be removable from frame 22 and may include one or more electrical connectors for establishing electrical communication between electronic components on or in footboard 12b and/or headboard 12a and other electronic components supported on or in frame 22. Such electrical connector(s) may include any one or more of the connectors disclosed in commonly assigned U.S. patent application Ser. No. 13/790,762, filed Mar. 8, 2013, by applicants Krishna Bhimavarapu and entitled PATIENT SUPPORT APPARATUS CONNECTORS, the complete disclosure of which is incorporated herein by reference. Other types of connectors may also be used.

In one embodiment, electrical connectors are provided for establishing an electrical link between a user interface 30 that is positioned on, or integrated into, footboard 12b and patient support apparatus 12. User interface may take on a variety of different forms, such as, but not limited to, a touch screen, a Liquid Crystal Display (LCD), a plurality of buttons, switches, knobs, or the like, or any combination of these components. As described in one or more of the referenced applications and patents, user interface 30 may allow a user to control the operation of patient support apparatus 12. The electrical connection between user interface 30 and patient support apparatus 12 may take on different forms, including a direct electrical cable that runs from footboard 12b to patient support apparatus 12. In another embodiment, footboard 12b include electrical connectors that electrically couple user interface 30 to circuitry supported on frame 22. This circuitry is further in electrical communication with a port (not shown) to which an electrical cable from patient support apparatus 12 may be inserted, thereby establishing an electrical link between user interface 30 and patient support apparatus 12. In still other embodiments, communication may be wireless. An example of such wireless communication is disclosed in commonly assigned, U.S. patent Application Ser. No. 13//802,992, filed Mar. 24, 2013, by applicants Michael Hayes et al. and entitled COMMUNICATION SYSTEMS FOR PATIENT SUPPORT APPARATUSES, the complete disclosure of which is hereby incorporated herein by reference.

Elevation adjustment mechanisms 20 are adapted to raise and lower frame 22 with respect to base 16. Elevation adjustment mechanisms 20 may be implemented as hydraulic actuators, electric actuators, or any other suitable device for raising and lowering frame 22 with respect to base 12. In the illustrated embodiment of FIG. 1, elevation adjustment mechanisms 20 are operable independently so that the orientation of frame 22 with respect to base 16 may also be adjusted and allow support apparatus 12 to tilt a patient supported on patient support apparatus 12 to either the Trendelenburg orientation, or the reverse Trendelenburg orientation. Patient support deck 24 provides a surface on which cushion 14 is positioned so that a patient may lie and/or sit thereon. Patient support deck 24 may be formed from a plurality of sections, some of which may be pivotable about generally horizontal pivot axes.

For further details of the general construction of any of base 16, elevation adjustment mechanisms 20, frame 22, patient support deck 24, headboard 12a, and/or footboard 12b may take on any conventional designs or other known designs, such as, for example, that are disclosed in commonly assigned, U.S. Pat. No. 7,690,059 issued to Lemire et al., and entitled HOSPITAL BED, the complete disclosure of which is incorporated herein by reference; or that disclosed in commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosure of which is also hereby incorporated herein by reference. For examples of a suitable cushion or mattress reference is made to U.S. Pat. No. 9,420,895 (P405E) and U.S. Pat. No. 9,468,307, U.S. Ser. No. 13/836,813, filed Mar. 15, 2013, entitled INFLATABLE MATTRESS AND CONTROL METHODS (143667.150316 (P400A)), which are commonly owned by Stryker Corp. Of Kalamazoo, Mich., and incorporated by reference herein in their entireties. It should be understood that the construction of any of cushion 14, base 24, elevation adjustment mechanisms 28, frame 30, patient support deck 32, headboard 34, and/or footboard 36 may also take on forms different from what are disclosed in the aforementioned patents.

Directional terms, such as “vertical,” “horizontal,” “top,” “bottom,” “upper,” “lower,” “inner,” “inwardly,” “outer” and “outwardly,” are used to assist in describing the disclosure based on the orientation of the embodiments shown in the illustrations. The use of directional terms should not be interpreted to limit the disclosure to any specific orientation(s).

The above description is that of current embodiments of the disclosure. Various alterations and changes can be made without departing from the spirit and broader aspects of the disclosure as defined in the appended claims, which are to be interpreted in accordance with the principles of patent law including the doctrine of equivalents. This disclosure is presented for illustrative purposes and should not be interpreted as an exhaustive description of all embodiments of the disclosure or to limit the scope of the claims to the specific elements illustrated or described in connection with these embodiments. For example, and without limitation, any individual element(s) of the described disclosure may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present disclosure is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

1. A cover for covering a patient support apparatus cushion, said cover comprising:
a top cover layer;
a lower cover layer configured to join with said top cover layer for enclosing a cushion there between; and
an ePTFE membrane between said top cover layer and said lower cover layer to form a liquid barrier between said top cover layer and the cushion.

2. The cover according to claim 1, wherein said ePTFE membrane is attached to said top cover layer.

3. The cover according to claim 1, wherein said ePTFE membrane is unattached to said top cover layer.

4. The cover according to claim 1, wherein said top cover layer comprises first and second layers, said first layer being formed of a material forming an exterior surface of said cover, and said ePTFE membrane secure to said second layer.

5. The cover according to claim 4, wherein said ePTFE membrane is laminated to said second layer.

6. The cover according to claim 1, further comprising an inner layer, said ePTFE membrane secure between said inner layer and said second layer.

7. The cover according to claim 1, wherein said ePTFE membrane is configured as a sleeve or a sock with at least two open sides for receiving the cushion therein.

8. A mattress for a patient support apparatus, said mattress comprising:
a cushion;
a top cover layer;
a lower cover layer configured to join with said top cover layer for enclosing said cushion there between; and
an ePTFE membrane between said top cover layer and said cushion to form a liquid barrier between said top cover layer and said cushion.

9. The mattress according to claim 8, wherein said ePTFE membrane is attached to said top cover layer.

10. The mattress according to claim 8, wherein said ePTFE membrane is unattached to said top cover layer.

11. The mattress according to claim 8, wherein said top cover layer comprises first and second layers, said first layer being formed of a material forming an exterior surface of said cover, and said ePTFE membrane secure to said second layer.

12. The mattress according to claim 11, wherein said ePTFE membrane is laminated to said second layer.

13. The mattress according to claim 8, wherein said ePTFE membrane is configured as a sock enclosing said cushion therein.

14. The cover or mattress according to claim 8, further comprising an intrusion indicator configured to indicate moisture or liquid intrusion through said cover, and said instruction detection configured to indicate a change in moisture, wetness, or pH.

15. The cover or mattress according to claim 14, wherein said intrusion indicator is secured said top cover layer.

16. The cover or mattress according to claim 15, wherein said intrusion indicator comprises an ink printed on or in said top cover layer.

17. The cover or mattress according to claim 15, wherein said intrusion indicator comprises a tape secured to said top cover layer between said top layer and the cushion.

18. The cover or mattress according to claim 17, wherein said tape extends exteriorly of said cover.

19. The cover or mattress according to claim 15, wherein said top cover layer includes a transparent window, and said intrusion indicator is located behind and viewable through said window.

20. The cover or mattress according to claim 14, wherein said intrusion indicator comprises a hydrochromic ink or hydrochromic tape.
